(19) United States
(12) Patent Application Publication
(10) Pub. No.: US 2002/0114788 A1
(43) Pub. Date: Aug. 22, 2002

(54) Cell implantation therapy for neurological diseases or disorders

(76) Inventors: Ole Isacson, Cambridge, MA (US); Kwang Soo Kim, Lexington, MA (US)

Correspondence Address: CLARK & ELBING LLP, 101 FEDERAL STREET, BOSTON, MA, 02110, US

(21) Appl. No.: 09/917,126

(22) Filed: Jul. 27, 2001

Related U.S. Application Data
(63) Continuation-in-part of application No. 09/626,677, filed on Jul. 27, 2000.

(51) Int. Cl. A61K 48/00; C12N 5/08

(52) U.S. Cl. 424/93.21; 435/368; 435/456

(57) ABSTRACT

Disclosed herein is a method for generating functional lineage-restricted progenitors from embryonic stem cells for obtaining donor cells of specific neuronal cell-fate, in sufficient quantities for the unmet cell transplantation need for treating patients with neurodegenerative diseases or disorders.

### CROSS REFERENCE TO RELATED APPLICATIONS

This application is a continuation in part of and claims priority from U.S. Pat. No. 09/626,677, filed Jul. 27, 2000, which is hereby incorporated by reference.

### STATEMENT AS TO FEDERALLY SPONSORED RESEARCH

This invention was sponsored in part by Grant #P50 NS39793-01 from the National Institutes of Health. This work was also sponsored in part by the following federal grant awards: Udall Parkinson's Disease Research Center of Excellance (P50 NS39793), DAMD17-98-1-8618 and DAMD17-99-1-9482. Support from the Kinetics Foundation and the Parkinson Alliance is also acknowledged. The Government has certain rights to this invention.

### BACKGROUND OF THE INVENTION

The field of the invention is cell implantation therapy for neurological disorders.

Neurodegenerative disorders such as Parkinson's , Alzheimer's , and Huntington's disease are becoming ever more prominent in our society. Additionally, many neurological disorders and diseases are associated with seratonergic or dopaminergic neurons. A direct approach towards therapeutic treatment of these diseases is through replacement therapy where normal tissue is transplanted back to the nervous system. Recently, significant progress has been achieved with transplants in Parkinson's disease (PD), but the process is heavily dependent on an unstable and problematic source of fetal tissue. Neural stem cells may become the tissue/cell source necessary for developing the therapeutic potential of neural transplantation. Stem cells are self-renewing, multipotent and provide a well-characterized and clean source of transplantable material to replace intrinsic neuronal systems, that do not spontaneously regenerate after injury, such as the dopaminergic (DA) system affected in PD and aging. Current clinical data indicate proof of principle for this cell implantation therapy for PD. Furthermore, the disease process does not appear to negatively affect the transplanted cells, although the patient's endogenous DA system degeneration continues.

To date, stem cells have been purified and characterized from several tissues. For example, neural stem cells have been purified from the mammalian forebrain (Reynolds and Weiss, Science 255:1707-1710, 1992) and these cells were shown to be capable of differentiating into neurons, astrocytes, and oligodendrocytes. PCT publications WO 93/01275, WO 94/16718, WO 94/10292 and WO 94/09119 describe uses for these cells. Neural stem cells may be used to generate oligodendrocytes and/or astrocytes for use in transplants for the treatment of multiple sclerosis and other myelin-associated diseases (Brustle et al., Science 285: 754 (1999)), or used to generate Schwann cells for treatment of spinal cord injury (McDonald et al., Nat. Med. 5: 1410 (1999)). The implementation of neural stem cell lines as a source material for brain tissue transplants is currently limited by the ability to induce specific neurochemical phenotypes in these cells (Wagner et al., Nat. Biotechnol. 17(7): 653, 1999). Specifically, there is a large unmet need for clinical cell implantation to patients suffering from neurological disorders such as PD and other neurodegenerative disorders. It would be very useful if there were accessible stem cells capable of differentiating into pure specific cell types, for example, DA neurons for clinical cell implantation to patients suffering from PD. Thus, what is required is a method for generating optimal cells for replacement, such as highly specialized human DA neurons that are capable of repairing an entire degenerated nigro-striatal system or homogeneous cells or defined heterogeneous cell populations that can be reliably obtained and generated in sufficient numbers for a standardized medically effective intervention.

### SUMMARY OF THE INVENTION

In general, the invention provides a method to generate functional lineage-restricted progenitors from embryonic stem cells for obtaining donor cells of specific neuronal cell-fate, in sufficient quantities for the unmet cell transplantation need for treating patients with neurological diseases or disorders; for example, DA neural cells for the transplantation therapy of PD. In particular, the invention features the selection of unmodified, totipotent embryonic stem cells derived from blastocysts, and inserting into these cells one or more cell-fate inducing genes, e.g., Nurr-1, PTX3, Phox 2a, AP2, Shh, that render them cell-fated to neurons.

The ES cells are capable of differentiating under appropriate conditions to DA neurons, serotonergic neurons, astrocytes, Schwann cells, and/or oligodendrocytes. From differentiated ES cells, homogeneous cell populations of specific neuronal cell-fate are isolated by inserting a selectable marker gene cassette into a cell-specific gene expressed in a specific neuronal cell-type. Homogeneous cells or defined heterogeneous cell populations that can be reliably obtained and generated in sufficient numbers for a standardized medically effective intervention are also featured in this invention. For example, inserting a selectable gene cassette, e.g., b-geo (encoding for both neomycin resistance and b-galactosidase) into the dopamine transporter (DAT) or the tyrosine hydroxylase (TH) gene allows the selective isolation of DA neurons. These pure DA neurons are a useful source of donor cells for grafts into PD patients. Likewise, one can isolate serotonergic neurons from differentiated ES cells by inserting the same b-geo gene cassette into the tryptophan hydroxylase or the serotonin transporter gene that is expressed by serotonergic neurons or isolate astrocytes by inserting the b-geo gene cassette into the fibrillary acidic protein gene expressed by astrocytes. Furthermore, other nerve cells or glial cells can be similarly targeted for lineage restricted populations derived from embryonic stem cells. Specific lineage-restricted neural precursors thus can be isolated and expanded as a pure population, and used as donor cells in transplantation therapy of different neurological diseases, disorders, or abnormal physical states. The stem cells may themselves be transplanted or, alternatively, they may be induced to produce differentiated cells (e.g., neurons, oligodendrocytes, Schwann cells, or astrocytes) for transplantation.

Accordingly, in a first aspect, the invention features a method of treating a human patient suffering from a neurodegenerative disease, including engrafting into a patient a population of ES recombinant cells that includes one or more cell fate-inducing genes that permit the cells to form neurons in the patient. Preferably, the cell fate inducing gene may be one or more of Nurr-1, PTX3, Phox 2a, AP2, and Shh. In one preferred embodiment, the one or more cell-fate inducing genes permit the cells to form DA neurons.

In a related aspect, the invention features a method of treating a human patient suffering from a neurodegenerative disease, wherein the cells are made by the steps of: a) obtaining one or more stem cells, b) transfecting one or more stem cells with one or more cell fate inducing genes, c) selecting one or more transfectants from step b), and d) expanding one or more selected transfectants from step c) to form a population of recombinant cells. Preferably, the step d) includes inducing cell division using a growth factor.

In another related aspect, the invention features a method of treating a human patient suffering from a neurodegenerative disease, wherein the cells are made by the steps of: a) obtaining one or more stem cells, b) expanding one or more stem cells, and c) transfecting multiple cells in the expanded cells from step b) with one or more cell fate inducing genes to form the population of recombinant cells. Preferably, step b) includes inducing cell division using a growth factor.

In preferred embodiments of each of the foregoing aspects of the invention, the cells are human unmodified, totipotent embryonic stem cells (TESCs). In other embodiments of the invention, the TESCs can be from, for example, non-human primates, mice, and rats.

In preferred embodiments of each of the foregoing aspects of the invention, the recombinant cells are a homogeneous cell population of a specific neuronal cell-type.

In preferred embodiments of each of the foregoing aspects of the invention, the one or more cell fate inducing genes cause the cells to form DA neurons. In other embodiments of the invention, the TESCs may, under appropriate conditions, differentiate into neurons, astrocytes, Schwann cells, and/or oligodendrocytes.

In preferred embodiments of each of the foregoing aspects of the invention, the growth factor used to expand the TESCs with or without the inserted genes for cell-fate induction is leukemia inhibitory factor (“LIF”). In other embodiments, a growth factor used to expand TESCs is basic fibroblast growth factor or epidermal growth factor.

TESCs can be stably or transiently transformed with a heterologous gene (e.g., one encoding a therapeutic protein, such as a protein which enhances cell divisions or prevents apoptosis of the transformed cell or other cells in the patient, or a cell fate-determining protein).

By “totipotent embryonic stem cell” or “TESC” is meant a cell that has the potential of differentiating into any type of cell. An embryonic stem cell is “totipotent” because it has the potential to differentiate into more than one cell type (e.g., a neuron, a skin cell, a hematopoietic cell).

The invention also features a pharmaceutical composition including (i) growth factor-expanded TESCs containing one or more cell-fate inducing genes, and (ii) a pharmaceutically acceptable carrier, auxiliary, or excipient.

Other features and advantages of the present invention will become apparent from the following detailed description and the claims. It will be understood, however, that the detailed description and the specific examples, while indicating preferred embodiments of the invention, are given by way of example only, and various changes and modifications within the spirit and scope of the invention will become apparent to those skilled in the art from this detailed description.

### BRIEF DESCRIPTION OF THE DRAWINGS

FIG. 1 is a diagrammatic representation of the steps for ES cell procedures including in vitro expansion, chemical or spontaneous induction into neurons after implantation into the adult brain. Totipotent embryonic stem cells derived from the inner cell mast of blastocyst are propagated in culture in the presence of leukemia inhibitory factor (LIF). Prior to transplantation, LIF is removed, and the cells are then treated with retinoic acid (A) or are transplanted directly (B) into adult brain.

FIG. 2 is a schematic representation of the steps involved in the non-linear trigger gene-induction of embryonic stem cells differentiating to donor neural cells, that are used for cell transfer/transplantation.

FIG. 3A is the vector map of pIRES2-EGFP and FIG. 3B is the vector map of pIRES2/EGFP/Nurr1 which expresses both the green fluorescent signal (EGFP) and dopamine-specific transcription factor Nurr1 .

FIG. 4 demonstrates the transcriptional activities of four different promoters in ES and 293T cell lines. FIG. 4A shows immunofluorescent staining in D3, J1 and 293T cells, and FIG. 4B is a graphical representation of relative luciferase activity in the three cell types transfected with luciferase expression constructs, as indicated.

FIG. 5 is an isolation and characterization of Nurr1-expressing cell lines. FIG. 5A is a reverse transcriptase polymerase chain reaction (RT-PCR) analysis of Nurr1 expressed from the EF promoter in 16 Nurr1 clones. FIG. 5B is immunohistological staining of in vitro differentiation of the Nurr1 clonal cells (Nb14) and the non-recombinant D3 cells. A much higher proportion of in vitro differentiated neurons (β-tubulin positive as indicated by the green color) are also TH positive (red) for the Nb14 clone, as compared to the naïve D3 cells after the same in vitro differentiation procedure.

FIG. 6 is an RT-PCR analysis of Nurr1 expresssion in stably transfected J1-rtTA cells. Two representative clones (#29 and #32) are shown.

FIG. 7 is a graph of mouse ES cell-associated restoration of DA dependent motor function in 6-OHDA lesioned rat striatum. Rotational behavior in response to amphetamine was tested pre-transplantation (pre TP) and at 5, 7, and 9 weeks post grafting. A significant decrease in absolute numbers of amphetamine-induced turning was seen in animals with ES cell neural DA grafts in the striatum (n=9) compared to control animals that received sham surgery (n=13).

### DETAILED DESCRIPTION

The present invention provides a method to generate functional lineage-restricted progenitors from embryonic stem cells for obtaining pure cell populations of specific neuronal cell-fate; for example, DA progenitors for obtaining donor DA neural cells in sufficient quantities for the unmet cell transplantation need for treating patients with neurodegenerative diseases or disorders. In particular, the invention features the selection of unmodified TESCs, and inserting these cells with one or more cell-fate inducing genes, e.g., Nurr-1, PTX3, Phox 2a, AP2, Shh, that render them cell-fated to neurons. The present invention also features methods of optimizing cell transplantation conditions, such as cell dilution and number of cells transplanted, in order to enhance differentiation to neural cell fate upon implantation in a subject. These TESC and TESC-derived cell transplant methods can induce specific neuronal cell fates.

TESCs under appropriate conditions differentiate into DA neurons, Schwann cells, oligodendrocytes and/or astrocytes and can serve as donor cells for transplants to treat neurodegenerative diseases, disorders, or abnormal physical states. For example, the cells may be used as a source of DA neurons for grafts into PD patients or seratonergic (5HT) neurons for patients suffering from other 5HT neuron-associated diseases such as depression. In one example, the cell-fate induction of TESCs results in differentiated DA neurons which may be implanted in the substantia nigra or striatum of a PD patient. In a second example, the cells may be used to generate oligodendrocytes and/or astrocytes under appropriate conditions for use in transplants for the treatment of multiple sclerosis and other myelin-associated diseases. In still another example, the TESCs may be used to generate Schwann cells for treatment of spinal cord injury. Using the genetic selection strategy as described in Example 7 infra, for example, specific neuronal cell-types can be isolated as a homogeneous population and used as donor cells in transplantation therapy of these different diseases. Alternatively, nearly homogenous cell populations, such as populations which are substantially homogenous (>75%, >90% or >95% pure) are featured in the invention. Heterogenous cell populations may be used in the methods of the invention, such as neural populations, monaminergic neural populations, or cell populations containing dopaminergic and seratonergic neurons, GABA neurons, or glial cells, for example. Furthermore, in any of the foregoing examples, the cells may be modified to express, for example, a growth factor or other therapeutic compound, if desired. We demonstrate that when low concentrations of ES cells in suspension in a pharmaceutically acceptable carrier, naïve ES cells differentiate to populations of cells that are predominantly dopaminergic and seratonergic neurons.

### CELL THERAPY

The TESCs of this invention may be used to prepare pharmaceutical compositions that can be administered to humans or animals for cell therapy. The cells may be undifferentiated or differentiated prior to administration. Dosages to be administered depending on patient needs, on the desired effect, and on the chosen route of administration.

The invention also features the use of the cells of this invention to introduce therapeutic compound(s) into the diseased, damaged, or physically abnormal CNS, PNS, or other tissue. The TESCs may thus act as a vector to deliver the compound(s). In order to allow for expression of other therapeutic compounds, suitable regulatory elements can be derived from a variety of sources, and may be readily selected by one of ordinary skill in the art. Examples of regulatory elements include a transcriptional promoter and enhancer or RNA polymerase binding sequence, and a ribosomal binding sequence, including a translation initiation signal. Additionally, depending on the vector employed, other genetic elements, such as selectable markers, may be incorporated into the recombinant molecule. The recombinant molecule may be introduced into the TESCs or the cells differentiated from the stem cells using in vitro delivery vehicles or in vivo techniques. Examples of delivery techniques include retroviral vectors, adenoviral vectors, DNA virus vectors, liposomes, physical techniques such as microinjection, and transfection such as via electroporation, calcium phosphate precipitation, or other methods known in the art for transfer of creating recombinant cells. The genetically altered cells may be encapsulated in microspheres and implanted into or in proximity to the diseased or damaged tissue. Protocols employed are well-known to those skilled in the art, and may be found, for example, in Ausubel et al., Current Protocols in Molecular Biology, John Wiley & Sons, New York, N.Y., 1997.

The methods of the invention can be used to treat any patient having a disease or disorder characterized by cell loss, cell deficiency or abnormality that can be ameliorated by administration of TESCs of the invention (or cells derived from these cells) to that patient. For example, TESCs may be used to generate DA neurons for use in transplants for the treatment of PD; oligodendrocytes and/or astrocytes for use in transplants for the treatment of multiple sclerosis and other myelin-associated diseases; Schwann cells for treatment of spinal cord injury; DA neurons and/or serotonergic neurons for treatment of other neurodegenerative diseases or disorders such as Alzheimer's , Huntington's and Hirschsprung's disease. For uses of stem cells, also see Ourednik et al. (Clin. Genet. 56: 267, 1999), hereby incorporated by reference.

Disorders and diseases associated with other neurological disorders such as psychiatric or mood disorders may also be treated with methods of the invention. Seratonergic and dopaminergic neurons are associated with, for example, such psychiatric disorders such as depression and schizophrenia

Optimization of transplantation conditions and procedures can have substantial effects on the cell fate of implanted ES cells. Transplantation of low concentrations of cells, and at low cell numbers, increases the number and type of nerve cells that develop from the ES cells upon implantation. Transplantation or cell implantation techniques may be adapted to particular subjects or patients. In rodents, for example, low cell numbers such as 200 or 2,000 embryonic stem cells transplanted into mice or rats result in grafts that largely become dopaminergic or seratonergic. By low numbers of cells is meant an amount of cells administered to a patient that minimizes graft cell-graft cell interactions, allowing optimization of graft cell-host cell interactions.

Suspensions of cells at low concentrations of implanted cells results in neural cell fate, and encourages development of particular neural lineages. Therapeutic concentrations of cells administered to a patient variously be 10, 20, 50, 100, 200, 300, 400, 500, 600, 800, 1000, 1200, 1400, 1600, 1800, 2000, 2500, 3000, 3500, 4000, 4500, 5000, 6000, or 7000 cells per microliter of a pharmaceutically acceptable carrier. Ranges of concentrations of cells in a carrier include, for example, 10-5000 cells/microliter, 10-1000 cells/microliter, 50-5000 cells/microliter, 50-2000 cells/microliter, 50-1000 cells/microliter 50-500 cells/microliter, 100-2000 cells/microliter, 100-1000 cells/microliter, etc. The number of cells grafted into a transplant site will also affect therapeutic efficacy. Transplanting low numbers of cells is featured in this invention. “Low numbers” in the methods of the invention would include less than or equal to 20,000, 15,000, 10,000, 8,000, 6,000, 5,000, 4,000, 3,000, 2,000, 1,000, 800, 600, 500, 400, 300, 200, 100, or 50 cells, for example.

Cell number and concentration of cells delivered in suspension would be optimized based on factors such as the age, physiological condition, and health of the subject, the size of the area of tissue that is targeted for therapy, and the extent of the pathology, for example. Transplantation conditions for various animals, including primates such as humans, would be optimized using the methods of this application. The transplant conditions of Examples 12-16 which have been optimized for rodents, would be similarly optimized to adapt to human physiology, as evident to one skilled in the art. Treatment of a human disorder affecting a larger region of the brain, for example, could require a larger number of cells to achieve a therapeutic effect similar to an effect of the graft on a smaller target region. Administration of cells to more than one site in a given target tissue is also featured in the invention, as multiple small grafts of low cell doses may facilitate induction of desired cell fates.

ES cell transplantation may be optimized by controlling the concentration of ES cells implanted in a subject, by controlling the total number of cells implanted, or by altering both variables. Additionally, complete or near complete dissociation of graft cells from each other prior to transplantation, such as to create a suspension of single cells, may affect neural fate. Implantation of ES cells as a single large bolus of 100,000-300,000 cells in a mature brain created conditions in which donor cells formed grafts with high cell densities in prior studies. We demonstrate that the numbers and dilution of total cells implanted in animal brains affects the cell fate of naive ES cells upon implantation.

Thus, experiments allowing implantation of fewer cells provide improved control over the differentiation process of these multi-potent ES cells into neuronal phenotypes, perhaps due to increased graft-host interactions.

Optimizing ES cell transplantation procedures to encourage the differentiation of the cell to particular cell fates, such as to maximize differentiation to neural cell fate, may be useful by itself or in combination with the recombinant ES cells described herein. This methodology for implantation of diluted ES cell cultures may similarly enable grafts of transgenic ES cells to be enriched for neural cells. Cell populations formed from grafted cells may be identified by assays for cell-specific markers, or for particular phenotypes. For example, various neurons will express cell specific proteins, or excrete specific factors. Neuronal cell fates may be analyzed with histological procedures, metabolic changes, electrical changes, pharmacological challenges, or functional or behavioral effects post implantation. In vivo imaging, for example, may be used to demonstrate restored neural functions.

Methods featured in the invention may also be optimized for naïve ES cells, or for cells that have been manipulated, such as to encourage differentiation to a particular cell fate or express a therapeutic factor. Such manipulations include altering culturing conditions, such as increasing or decreasing levels of factors that influence differentiation or development to one or more particular cell fates. It may be preferable for particular uses to implant low cell numbers or low density functional lineage-restricted progenitors or cells derived from such cells. Cell fate inducing genes or therapeutic factors may be expressed in ES cells used in these transplant methods. By way of example, Nurr 1 expressing transgenic cells may be induced to develop primarily or exclusively into dopaminergic neurons upon implantation. Such cells may be induced to develop into homogenous or near homogeneous cell populations upon implantation by a combination of manipulation of the ES progenitors and alteration of transplant conditions.

Transgenic ES cells capable of expressing a heterologous gene may express cell fate-associated genes or they may produce therapeutic factors. Homogeneous, or near homogeneous populations of cells may be preferred, such as purely domaminergic, seratonergic, noradrenergic, GABA, or cholineacetyltransferase (ChAT) nerve cells. Alternately, directed development of ES cells to particular heterogenous cell fates may be preferred, such as the predominantly dopaminergic and seratonergic neuron populations described in Example 9, below. Heterogeneous populations of implanted cells which are specific, defined, and therapeutically active can be induced by methods of the invention. Such heterogenous populations could be neural or glial, including combinations of monoaminergic, dopaminergic, seratonergic, noradrenergic, cholinacetyltransferase, or GABA neurons, for example.

Positive and negative regulators of neuronal fate and differentiation to particular lineages are known in the art. ES cells of the invention may be manipulated to express or select for cells expressing such regulatory factors. The application of low doses of ES cells resulted in neuronal DA containing grafts consistent with the theory of neuronal fate as a default pathway. During early development, ectodermal cells in the developing embryo either become epidermal or neural. Certain regions like the Spemann organizer in amphibians and the Node in mice have important roles in the induction of neurons from the ectoderm. (Zhou, et al. Nature 361, 543-547(1993)) Molecules such as noggin, follistatin, Xnr 3, cerberus and chordin are secreted from the Spemann organizer and are thought to be responsible for the neuralizing effect. (See, e.g., Smith et al. Cell 70, 829-840 (1992); Hemmati-Brivanlou et al. Cell 77, 283-295 (1994); Hansen et al., Development 124, 483-492 (1997); Piccolo et al., Nature 397, 707-710 (1999); Sasai et al. Cell 79, 779-790 (1994); Lamb et al., Science 262, 713-718 (1993); and Sasai et al., Nature 376, 333-336 (1995)). Bone morphogenetic protein 4 (BMP-4) is a powerful inductor of epidermis and an inhibitor of neural fate. (Wilson and Hemmati-Brivanlou, Nature 376, 331-333 (1995)). Disruption of BMP signaling by introduction of dominant-negative versions of these factors or their receptors can lead to neural induction and ectopic neural tissues can be induced in developing mouse embryos after heterotopic grafting of the node. (See, e.g., Sasai, Nature, supra; Hawley et al., Genes Dev 9, 2923-2935 (1995); Xu et al., Biochem Biophys Res Commun 212, 212-219 (1995); and Beddington, Development 120, 613-620 (1994)). Recently, Tropepe et al. showed that dilution of ES cell concentration in vitro facilitates neuronal differentiation compared to ES cell cultures of higher density. (Tropepe et al. Neuron 30, 65-78 (2001)). They also showed that this effect can be mimicked by BMP antagonists such as noggin and cerberus as well as by using ES cells with a targeted null mutation in the Smad4 gene, which is a critical intracellular transducer of multiple TGF-β signaling pathways. Furthermore, graft location does not seem to be important for neuronal phenotype differentiation, since similar graft composition is found for grafts located in the striatum, kidney capsule, midbrain, thalamus and cortex. This is in contrast to adult or non-ES cell precursors or adult stem cells that differentiate into glial cells in the cerebellum or striatum (but not neurons as in our study).

### EXAMPLE 1

TESC preparation

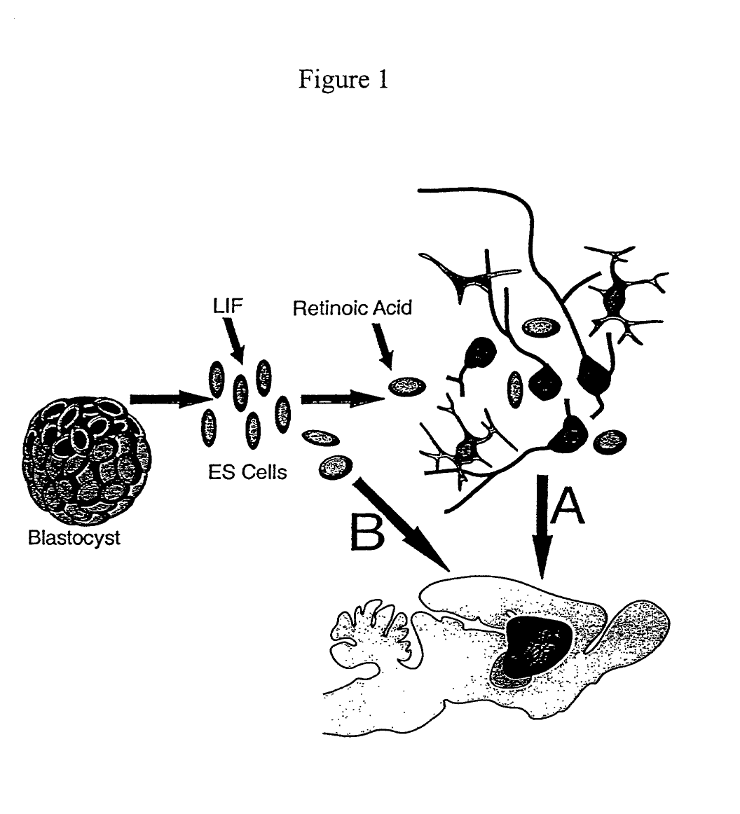

The mouse blastocyst-derived embryonic stem (ES) cell lines D3 and E14TG2a (A.T.C.C.; Rockland, Md.) and B5 (Hadjantonakis et al., Mech. Dev. 76: 79 (1998) were used for all studies ( Doestschman et al., J. Embryol. Exp. 87: 27-45, 1985; Finger et al., J. of Neurol. Sci. 86: 203-213); the E14TG2a line was HPRT-deficient. All ES cell lines were propagated and maintained as described (Deacon et al., Experimental Neurology 149: 28 (1998)). Undifferentiated ES cells were maintained on gelatin coated dishes in Dulbecco's modified Minimal Essential Medium (DMEM, Gibco/BRL, Grand Island, N.Y.) supplemented with 2 mM glutamine (100× stock from Gibco/BRL), 0.001% β-mercaptoethanol, 1× non-essential amino acids (100× stock from Gibco/BRL), 10% donor horse serum (HyClone, Logan, Utah), and human recombinant leukemia inhibitory factor (LIF; R & D Systems, Minneapolis, Minn.) (Abercrombie, M. Anat. Rec. 94, 239-247 (1946)). Early passage cultures were frozen (90% horse serum/10% DMSO), thawed for use, and cultured for two weeks in the presence of LIF. Cells were trypsinized (0.05% trypsin-EGTA; GIBCO), resuspended, then seeded at 1.5×10⁶ cells in 5 ml of DMEM +0.5 mM retinoic acid (RA+) (Sigma Chemical Co., St. Louis, Mo.) or in the same media without RA (RA−) in a 60 mm Fisher brand bacteriological grade petri dish, in the absence of LIF. Horse serum was replaced by 10% fetal calf serum (FCS; Hyclone) during this treatment. ES cells did not adhere to the dish but formed small aggregates (embryoid body). After 2 days of incubation at 37° C., the cells were transferred to a 15 ml sterile culture tube and allowed to settle, and the media was replaced with an equal volume of fresh RA+ or RA− media. The cells were then re-plated and incubated for an additional 2 days. After 4 days, cells were collected and rinsed once in Ca²⁺and Mg²⁺-free Dulbecco's Phosphate-Buffered Saline (D-PBSa, Gibco/BRL). D-PBSa was removed, 0.5 ml of trypsin solution was added, and the cells were incubated for 5 minutes at 37° C., then triturated with a pasteur pipette to dissociate the cells. The trypsin solution was replaced with 0.1 M phosphate buffered saline pH 7.4 (PBS), and viability was determined by the acridine orange-ethidium bromide method (Brundin, P., et al., Brain Res. 331, 251-259 (1985)); viability of cells after removal from the culture dish was greater than 95% in all cases. ES cells derived directly from monolayers after LIF removal were also implanted in some cases, following the above procedures minus the incubation steps. No systematic difference due to incubation time was observed in the resulting grafts and so RA− cases are pooled in this report (see FIG. 1 for schematic showing basic steps for ES cell procedures).

### EXAMPLE 2

Genetic modification of mouse blastocyst-derived ES cells

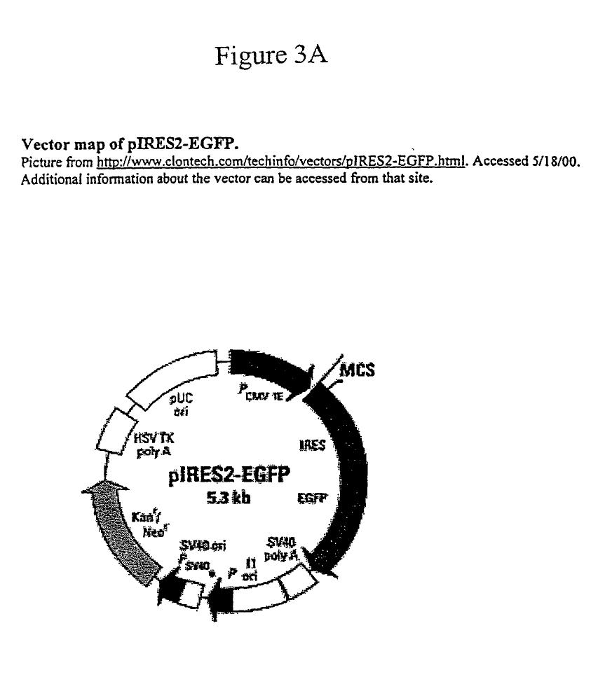

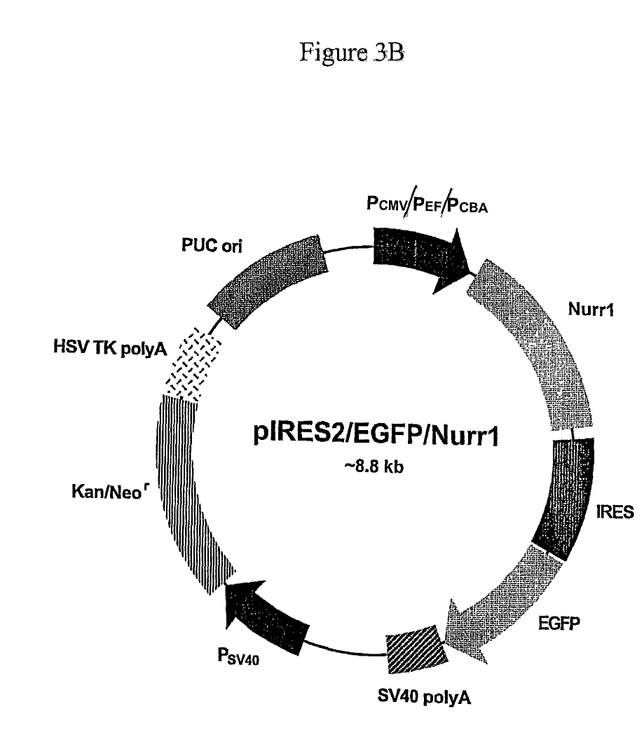

By way of example, construction of a Nurr1 expressing ES cell line is described. Nurr1 cDNA was subcloned into the SacI site in pIRES2-EGFP (Clontech)[see FIGS. 3A and 3B]. Nurr1-containing plasmids were amplified in E. coli and purified with the QIAGEN plasmid purification kit (QIAGEN Inc.). The construct's functionality was tested by demonstrating its ability to induce tyrosine hydroxylase (TH) reporter gene expression in cell lines such as BE(2)C cells, followed by β-galactosidase and CAT-assays. pIRES2-EGFP with [see FIG. 3B] and without Nurr1 insert [see FIG. 3A] was linearized with Afl II and isolated after 1% agarose gel electrophoresis for transfection to embryonic stem (ES) cells.

ES D3 cells were seeded into gelatin coated dishes to an approximate confluence of 25%. Next morning, the cells were transfected using Lipofectamin PLUS (GIBCO BRL, Life technologies, Gaithersburg, Md., USA) according to the manufacturer's protocol. [30 μg DNA in 750 μl serum free media and 60 μl PLUS were mixed an incubated at RT for 15 minutes after which 60 μl Lipofectamin in 750 μl serum free media was added and the mixture incubated for another 15 minutes at RT. The mixture was added drop-wise to cultured cells in a 100 mm dish containing 5 ml ES-media (450 ml high glucose DMEM, 50 ml horse serum (HS), 5 ml 100× L-glutamine, 5 ml Hees, 5 ml 100× NEAR, 5 ml β-mercaptoethanol and 1001. LIF 30 μg/ml).]

After 24th, 5 ml fresh ES-media was added and after another 6th cells were split and cultured in ES media containing 500 μg/ml Neomycin (G418 Sulfate, Clontech Palo Alto, Calif., USA) for selection. Leftover cells were frozen in ES-freezing media (90% horse serum and 10% DMSO). The concentration of Neomycin needed for selection was determined by culturing untransfected and transfected cells in a range of titers of Neomycin.

Cells split 30 h after transfection were pooled together, cell stocks were lo made, and cells were cultured to be used for RT-PCT analysis and immunocytochemistry. Fresh transfected cells (frozen 30 h after transfection) were thawed and seeded, highly diluted, in gelatin coated dishes and grown for five days in ES-media with G418 (500 μg/ml). Well-isolated colonies were picked using cloning cylinders and cloning discs and transferred to a gelatin coated 24 well plate. Cells were grown to confluency (between 10 and 14 days), harvested and frozen in 0.5 ml ES-freezing media. A small number of the cells (˜1/8) were expanded for RNA preparation. Clones were screened to detect Nurr1-expression, using GeneAmp Thermostable rTth Reverse Transcriptase RNA PCT Kit (PERKIN ELMER, Branchburg, N.J., USA) according to the manufacturer's protocol.

Multiple Nurr1-expressing ES cell lines isolated after Neomycin selection were used for in vivo transplantation as well as in vitro differentiation into the DA phenotype. Differentiation of neural stem cells into DA neurons requires overexpression of Nurr1 as well as a factor derived from local type 1 astrocytes (see Wagner et al., Nat. Biotechnol. 17(7): 653, (1999)). Hence, these Nurr1 expressing ES cells can also serve as a source of DA neurons. Protocols employed here are well-known by those skilled in the art and may be found, for example, in Ausubel et al., Current Protocols in Molecular Biology, John Wiley & Sons, New York, N.Y., 1997.

These non-human primate ES cell lines provided an accurate in vitro model for human transplantation studies.

### EXAMPLE 3

In vitro differentiation of naïve and transgenic ES cell lines The method of differentiating ES cells into neural progenitor cells and into DA and serotonergic neurons in vitro has been reported (Lee et al., Nat. Biotechnol. 18: 675, (2000)). This procedure was adapted for D3 and B5 ES cells and further modified for Nurr1-expressing transgenic ES cell lines. Briefly, D3 and B5 ES cells were differentiated into embryoid bodies (EBs) in suspension culture for four days after removal of leukemia inhibitory factor (LIF). The EBs are then plated onto adhesive tissue culture surface in the ES cell differentiation medium. After 24 hr of culture, nestin-positive cells were selected by replacing the medium by serum-free ITSFn medium (Rizzino and Crowley, Proc. Natl. Acad. Sci. 77: 457, (1980)); Okabe et al., Mech. Dev. 59: 89, (1996)). After 6-10 days of selection, nestin-positive cells were expanded by dissociating the cells by trypsinization and subsequent plating on tissue culture plastic containing N2 medium (Johe et al., Genes Dev. 10:129, (1996)) supplemented with laminin (1 mg/ml) and bFGF (10 ng/ml). After expansion for six days, the medium was changed every two days. Differentiation was induced by removal of bFGF from the medium. Signaling molecules known to induce the TH+ phenotype, e.g., analog of cAMP, retinoic acid, Shh, FGF8, and ascorbic acid (Kalir and Mytilineou, J. Neurochem. 57: 458, (1991); Kim et al., Proc. Natl. Acad. Sci., (1993); Lee et al., Nat. Biotechnol. 18: 675, 2000) were used and compared in naive and transgenic ES cell lines. Expression of marker expression was examined by immunocytochemistry and RT-PCR analysis. To determine the molecular changes between nestin-positive neural progenitor cells and more differentiated TH+neurons, EBs were collected from each stage of in vitro differentiation as described above. Poly (A)+RNA were isolated and the probes prepared subsequently.

### EXAMPLE 4

ES cell transplantation

Sprague-Dawley rats (300-350 g) and C57/B15 mice (14-17 g) (Charles River Labs, MA) were used as intracerebral-transplant recipients. Cell concentrations and dosages varied in different experiments: rat hosts received from 100,000 to 300,000 viable ES cells per right striatum (60,000-100,000 viable cells/1.), and mice received 60,000 ES cells per right striatum (60,000 viable cells/1.). For all neural surgical procedures, animals were anesthetized with pentobarbital (65 mg/kg, i.p.), and placed in a Kopf stereotaxic frame (with Kopf mouse adapter for mice). Mice (n=7) used as intracerebral transplant hosts were normal adult females, and rats (n=31) used as transplant hosts were adult females that had received prior unilateral nigrostriatal 6-hydroxydopamine (6-OHDA) lesion removing at least 97% of DA innervation, as previously described (Galpem et al., Cell Transplant. 140 :1-13, (1996)). ES cells were implanted stereotaxically (from Bregma: A+1.0 mm, L −2.5 mm, V −4.5 mm; IB −2.5 mm). A 101. Hamilton syringe attached to a 22S-gauge needle (ID/OD 0.41 mm/0.71 mm) was used to deliver 11. (mouse) or 3-51. (rat) of ES cell suspension (rate: 1 ml/min, allowing an additional 2 min for the final injection pressure to equilibrate before slowly withdrawing the injection needle). Starting on the day prior to transplantation, rats were immunosuppressed with Cyclosporine-A (CsA, Sandimmunne, Mass.)(10-15 mg/kg, s.c. daily) diluted in extra virgin olive oil for the duration of the experiment to prevent graft rejection. CsA blood levels were assayed each week (Quest Diagnostics, MA).

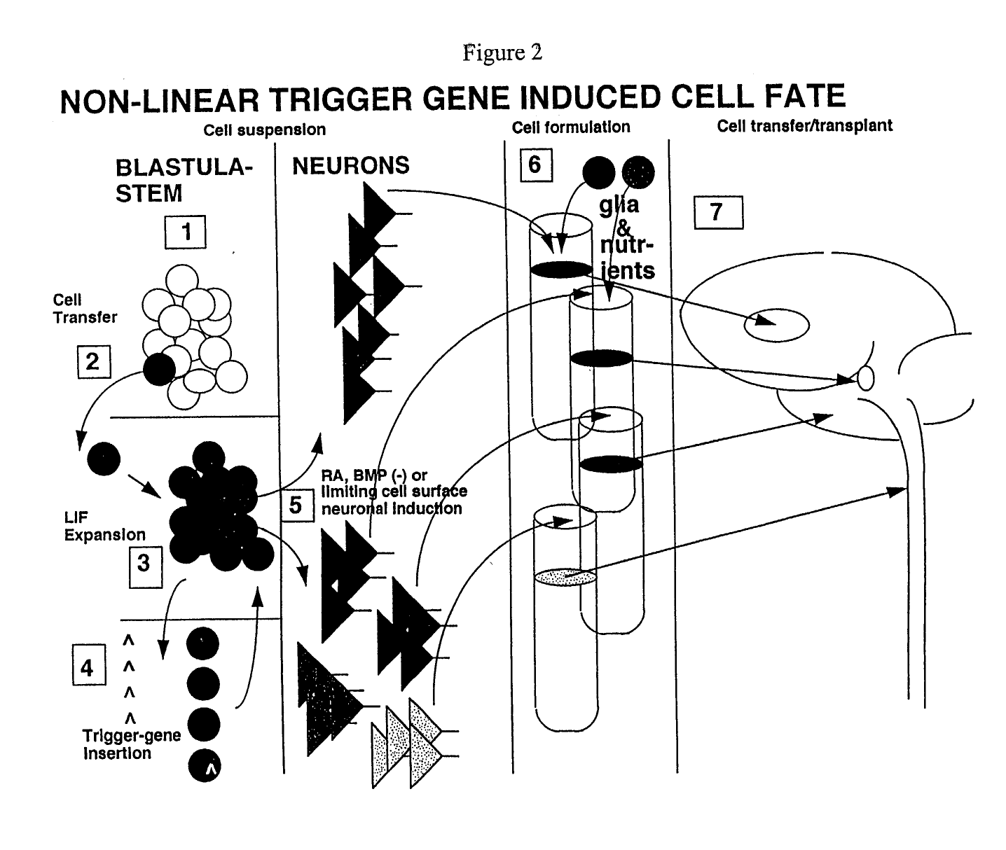

Mice were not immunosuppressed. Nude mice (Charles River) were used as kidney-capsule transplant recipients. Mice were anesthetized (as above), and 50,000 ES cells (in 1 ml), not pre-treated with RA, were injected into a blood clot derived from host blood; this clot was then implanted unilaterally into one kidney capsule (n=3 with E14TG2a line and n=3 with D3 line). (See FIG. 2 for schematic showing the various steps involved in the non-linear gene induction of embryonic stem cells differentiating to donor neural cells that are used for transplantation)

Histological procedures

Two or four weeks after transplantation, animals were terminally anesthetized (pentobarbital; 100 mg/kg), then perfused intracardially with 100 ml heparin saline (0.1% heparin in 0.9% saline), followed by 400 ml of paraformaldehyde (4% in PBS). The brains or kidney capsules were removed and post-fixed for 8 hours in the same 4% paraformaldehyde solution. Following post-fixation, the brains and kidney capsules were equilibrated in sucrose (30% in PBS), sectioned (40 mm) on a freezing microtome, and collected in PBS. Sections were divided into 6-8 series and stored in PBS at 4 C. Separate series were processed for either Niss1 staining (cresyl violet acetate), or acetylcholinesterase (AChE) histochemistry (as described in Pakzaban et al., Exp. Brain Res. 97: 13-22). Immunohistochemical markers used for tissue processing included antibodies directed against neuron-specific enolase (NSE, Dako, Carpenteria, Calif.), mouse-specific Thy 1.1 (Clone TN-26, Sigma), tyrosine hydroxylase (TH; PelFreez, Rogers, Ak.), 5-hydroxytryptamine (5-HT, Amel Products, New York, N.Y.), 200 kD+68 kD neurofilament (NF, Biodesign, Kennebunkport, Me.), dopamine-β-hydroxylase (DβH; Chemicon, Temecula, Calif.), proliferating cell nuclear antigen (PCNA; Chemicon), and glial fibrillary acidic protein (GFAP: Boehringer-Mannheim).

Free floating tissue sections were pretreated with 50% methanol and 3% hydrogen peroxide in PBS for 20 minutes, washed 3 times in PBS, and incubated in 10% normal goat serum (NGS) in PBS for 60 minutes prior to overnight incubation on a shaking platform with the primary antibody. After a 10-minute rinse in PBS and two 10-minute washes in 5% NGS, sections were incubated in biotinylated secondary antibody (goat-anti-rabbit or goat-anti-mouse, depending on primary species) at a dilution of 1:200 in 2% NGS in PBS at room temperature for 60-90 min. The sections were then rinsed three times in PBS and incubated in avidin-biotin complex (Vectastain ABC Kit ELITE; Vector Labs) in PBS for 60-90 min at room temperature. Following thorough rinsing with PBS and Tris-buffered saline, sections were developed for 5-30 min in 0.04% hydrogen peroxide and 0.05% 3,3′-diaminobenzidine (Sigma) in Tris-buffered saline. Controls with omission of the primary antibody were performed on selected sections to verify the specificity of staining. After immunostaining, floating tissue sections were mounted on glass slides, coverslipped, and analyzed with bright and darkfield light microscopy using a Zeiss Axioplan microscope. Quantitative analyses were performed with the aid of NIH Image software (Ray Rasband, NIH, Bethesda, Md.) and cell counts from serial sections were corrected and extrapolated for whole graft volumes using the Abercrombie method (Finger, S., et al., Journal of Neurological Sciences 86, 203-213 (1988). Selected images were digitized using a Leaf Lumina video scanning camera (Leaf Systems, Newton, Mass.) into Adobe Photoshop which was used to prepare and print final figures.

### EXAMPLE 5

Embryonic stem cell lines derived from human blastocysts

Fresh or frozen cleavage stage human embryos, produced by in vitro fertilization (IVF) were cultured to the blastocyte stage in G1.2 and G2.2 medium. These embryos were donated by individuals after informed consent and after institutional review board approval. 14 inner cell masses were isolated by immunosurgery, with a rabbit antiserum to BeWO cells, and plated on irradiated (35 grays gamma irradiation) mouse embryonic fibroblasts. Culture medium consisted of 80% Dulbecco's modified Eagle's medium (no pyruvate, high glucose formulation; Gibco-BRL) supplemented with 20% fetal bovine serum (Hyclone), 1 mM glutamine, 0.1 mM β-mercaptoethanol (Sigma), and 1% nonessential amino acid stock (Gibco-BRL). After 9-15 days, the inner cell mass-derived outgrowths were dissociated into clumps either by exposure to Ca²⁺/Mg²⁺free phosphate-buffered saline with 1 mM EDTA, by exposure to dispase, or by mechanical dissociation with a micropipette and replated on irradiated mouse embryonic fibroblasts in fresh medium. Individual colonies with a uniform undifferentiated morphology were individually selected by micropipette, mechanically dissociated into clumps, and replated. Once established and expanded, cultures were passaged by exposure to type IV colllegenase (1 mg/ml; Gibco-BRL) or by selection of individual colonies by micropipette. Clump sizes of about 50-100 cells were optimal. The resulting cells had a high ratio of nucleus to cytoplasm, prominent nucleoli, and a colony morphology similar to that of rhesus monkey ES cells. Cell lines can be cryopreserved and thawed when required. Continuous culturing does not lead to a period of replicative crisis in the cell lines (For details, see Thompson et al., Science 282 (5391): 1145 (1998), incorporated herein by reference). Also see Vescovi et al., J. Neurotrauma 16(8): 689 (1999); Vescovi et al., Exp. NeuroL, 156(1): 71 (1999); Brustle O et al., Science 285(5428): 754 (1999) for methods for isolation and /or intracerebral grafting of non-transformed embryonic human stem cells.

### EXAMPLE 6

Transformation of human TESCs

In therapy for neurodegenerative diseases, it is desirable to transplant cells that are genetically modified to survive the insults that caused the original neurons to die. In addition, TESCs may be used to deliver therapeutic proteins into the brain of patients with neurodegenerative disorders to inhibit death of host cells.

According to the invention, TESCs are induced to differentiate into a desired cell type by transfecting the cells with nucleic acid molecules encoding proteins that regulate cell fate decisions (e.g., transcription factors such as Nurr-1, PTX3, Phox2a, AP2, and Shh). Nurr1 is known to regulate the development of midbrain dopaminergic neurons (Zetterstrom et al., Science 276: 248, (1997)). Our studies further indicated that Nurr1 may control dopaminergic fate by directly transactivating TH gene transcription. Ptx3 is another transcription factor specifically expressed in dopaminergic neurons but its precise function is not clear as yet (Smidt et al., Proc. Natl. Acad. Sci. 94:13305, (1997); Smidt et al., Nat. Neurosci. 3: 337, (2000)). Recent studies have showed that Phox2a is critical for both the development and neurotransmitter identity of noradrenergic neurons (Morin et al., Neuron 18: 411, (1997); Yang et al., J of Neurochem. 71:1813, (1998)). Shh is a signaling molecule which has been shown to be critical for determining the development of both the dopaminergic and serotonergic neurons (Ye et al., Cell 93: 755, (1998)). Our recent analysis also indicated that AP2 may control both the TH and dopamine β-hydroxylase promoter activities and thus regulate catecholamine production. Using such a method, it is possible to induce the differentiation of the specific cell types required for transplant therapy. Recombinant adenoviral vectors can be used to manipulate both postmitotic sympathetic neurons and cortical progenitor cells, with no cytotoxic effects.

Blastocyst-derived TESCs were transfected with a recombinant, attenuated adenovirus carrying the β-galactosidase reporter gene inserted in the deleted E1 region. Multiplicity of infection (MOI) was calculated based on titration on cells for adenovirus-based vectors, and represents the number of plaque-forming units added per cell. Staining for expression of the β-galactosidase marker gene was performed. Cells were fixed with 0.2% glutaraldehyde in PBS (pH 7.4) for 15 minutes at 4° C. After two washes with PBS, cells were incubated for 18 hours in X-gal stain (2 mM MgCI₂, 1 mg/ml X-gal, 5 mM K₃Fe(CN)₆, and 5 mM K₄Fe(CN)₆ in PBS (pH 7.4). To estimate the percentage of cells that were infected, the total cell number and lacZ-positive cells can be counted in five random fields.

Similar Adenovirus vectors, carrying different regulatory cell-fate inducing genes including Nurr1, PTX3, Phox2a, AP2, and/or Shh, are constructed and used to express their gene products in TESCs. Expression of these genes is monitored by Northern Analysis, Western Analysis and/or Immunohistochemical analysis. Protocols for the same may be found, for example, in Ausubel et al., Current Protocols in Molecular Biology, John Wiley & Sons, New York, N.Y., 1997 and in Antibodies: A Laboratory Manual (E. Harlow and D. Lane, Cold Spring Harbor Laboratory, cold Spring Harbor, N.Y., 1988). Details of the cell-fate inducing genes can be accessed at: http://wwww.ncbi.nlm.nih.gov/Pubmed/: The National Center for Biotechnology Information; see below for Genebank Accession Numbers.

| Cell-fate inducing gene | Genebank accession number |
|---|---|
| Shh(human) | NM 000193 |
| NP-2(human) | X77343 |
| Phox2a(human) | NM 003924 |
| Phox2a1(human) | NM 005169 |
| PTX3(Rat) | AJOl 1005 |
| PTX3(human) | X6306 |
| Nurr1(human) | AB017586 |
| Nurr1(Rat) | U72345 |
| Nurr2(Mouse) | AB014889 |

### EXAMPLE 7

Selection of homogeneous cell populations of specific neuronal cell-fate from differentiated ES cells

ES cells can differentiate into various cell types in vitro by exposure to different extracellular signaling molecules. By combining several signaling molecules known to induce the DA neuronal cell-fate, a recent study reported that more than 20% of the cell population were induced to differentiate into tyrosine hydroxylase (TH)-positive cells (see Lee et al., Nat. Biotechnol. 18: 675 (2000)). However, these cell populations still contained various other different cell-types including serotonergic neurons and glial cells. At present, it is uncertain whether these mixed population of ES-derived cells are an optimal source of donor cells in transplantation therapy. Hence, we developed a strategy to selectively isolate homogenous cell populations with specific neuronal cell-fate; in particular, the DA cell-fate. A recent study showed that neuroepithelial cells can be efficiently selected from differentiated ES cells by inserting a selectable marker gene into the Sox2 gene that is specifically expressed in neuroepithelial cells (Li et al., Curr. Biol. 8:971 (1998)).

For DA neurons, dopamine transporter (DAT) is another specific marker protein in addition to that of TH. Introduction of a selectable marker/reporter gene cassette into the DAT or TH gene of ES cells allows the selective isolation of a homogenous cell population of DA neurons. Similarly, one can isolate a pure population of serotonergic neurons by inserting the selectable gene cassette into the tryptophan hydroxylase or serotonin transporter gene. This selection strategy can be employed in other cell-types, by introducing the selectable gene cassette into a gene known to be expressed in specific neuronal cell-types (e.g., the glial fibrillary acidic protein gene for isolating astrocyte cells).

Thus, to isolate the desired lineage-specific neural progenitors, plasmid constructs will be made in which the bifunctional selection marker/reporter gene cassette β-geo [coding for both the β-galactosidase and the neomycin resistance gene; see Friedrich G and Soriano P, Genes Dev. 5: 1513, (1991)] will be cloned into the cell-specific gene of interest in ES cells, such that the β-galactosidase and the neomycin phosphotransferase genes are expressed in a cell-specific manner. At the 3′ end of the cell-specific gene, a phosphoglycerate kinase-hygromycin (pGK-hygro) resistant gene will be cloned (see Mortensen RM et al., Mol. Cell. Biol. 12:2391, (1992)). The plasmid will be cut with restriction enzymes to linearize a fragment containing the 5′ region of the cell-specific gene β-geo cassette-pGK-hygro cassette-3′ sequence of the cell-specific gene. The linearized fragment will be electroporated into ES cells (see Klug MG et al., J. Clin. Invest. 98 :21, (1996); Li ML et al., Curr. Biol. 8: 971, (1998). Transfected clones will be selected by growth in the presence of 200 μg/ml hygromycin (Calbiochem, La Jolla, Calif.). Transfected ES cells will be cultured (see Smith AG et al., J Tissue Culture Methods 13: 89, (1991)) in Dulbecco's modified Eagle's medium (DMEM) (GIBCO/BRL, Grand Island, N.Y.) containing 10% fetal bovine serum (FBS) (GIBCO/BRL), 1% nonessential amino acids (GIBCO/BRL), 0.1 mmol/12-mercaptoethanol (GIBCO/BRL), 1 mmol/1 sodium pyruvate, 100 IU/ml penicillin, and 0.1 mg/ml streptomycin. The undifferentiated state will be maintained by 1,000 U/ml recombinant leukemia inhibitory factor (LIF) (GIBCO/BRL). To induce differentiation, hygromycin resistant ES cells will be plated onto a 100-mm bacterial Petri dish containing 10 ml of DME lacking supplemental LIF. After 3 d in suspension culture, the resulting embryoid bodies will be plated onto plastic 100-mm cell culture dishes and allowed to attach. The differentiated cultures will be grown in the presence of G418 (200 μg/ml;Gibco Laboratories, Grand Island, N.Y.), resulting in selection of cell-specific ES cells. Expression of cell-specific genes is monitored by Northern Analysis, Western Analysis and/or Immunohistochemical analysis. Protocols for the same may be found, for example, in Ausubel et al., Current Protocols in Molecular Biology, John Wiley & Sons, New York, N.Y., 1997 and in Antibodies: A Laboratory Manual (E. Harlow and D. Lane, Cold Spring Harbor Laboratory, cold Spring Harbor, N.Y., 1988). Details of the cell-specific genes can be accessed at: http://www.ncbi.nlm.nih.gov/Pubmed/: The National Center for Biotechnology Information; see below for Genebank Accession Numbers.

| | | Genebank |
|---|---|---|
| Neuronal cell-type | Cell-specific gene(human) | accession number |
| DA neurons | dopamine transporter(DAT) | D88570 |
| DA neurons | tyrosine hydroxylase(TH) | D00292 |
| serotonergic neurons | tryptophan hydroxylase | X83213 |
| serotonergic neurons | serotonin transporter | AF117826 |
| astrocytes | glial fibrillary acidic protein | BE222981 |

### EXAMPLE 8

Optimization of expression of a heterologous gene in ES cells

To optimize expression of cell fate inducing genes or therapeutic factors, the expression driven by various promoters was examined in undifferentiated and differentiated ES cells using expression constructs containing different cellular and viral promoters. The strength of different promoters was compared by generating expression vectors that drive expression of the reporter luciferase gene under the control of different promoter systems. Four promoters, CMV, elongation factor (EF), phosphoglycerate kinase (PGK) and chicken β-actin (CBA) promoters, were subcloned into pIRES-hrGFP vector (Stratagene). Each of these four constructs was transfected into D3 cells, and cells were fixed and analyzed by fluorescent microscopy 36 hours after transfection.

The plasmids were constructed as follows. pIRES-hrGFP was purchased from Stratagene. For pEF-hrGFP plasmid, EF1α promoter was PCR amplified from pTracer-CMV2 (Invitrogen) using primers containing NsiI or NotI linker for each ends, and digested with NsiI and NotI, and ligated into NsiI and NotI sites of pIRES-hrGFP vector. For pPGK-hrGFP plasmid, PGK promoter (EcoRV-BamHI fragment) from pRRL.PGK.GFP.Sin-18 (a gift from Dr. R. Zufferey at University of Zeneva, Switzerland) was ligated into NsiI and BamHI sites of pIRES-hrGFP vector. For pCBA-hrGFP plasmid, chicken b-actin promoter with CMV enhancer (SalI-EcoRI fragment) from pCX-EGFP (a gift from Dr. M. Okabe and Dr. J. Miyazaki at Osaka University, Osaka, Japan.) was ligated into NsiI and EcoRI site of pIRES-hrGFP vector. All constructs were confirmed by restriction digestion and sequencing analysis.

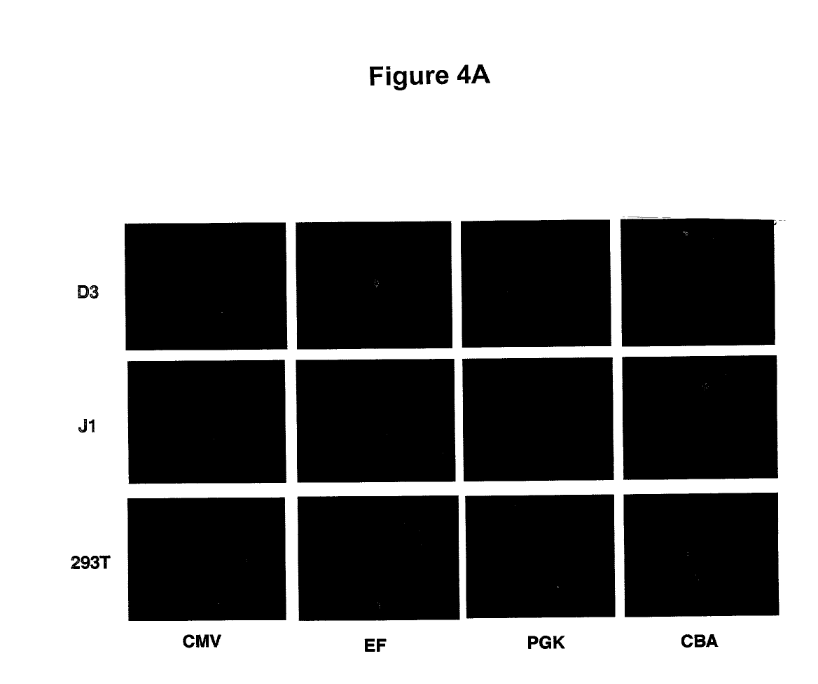

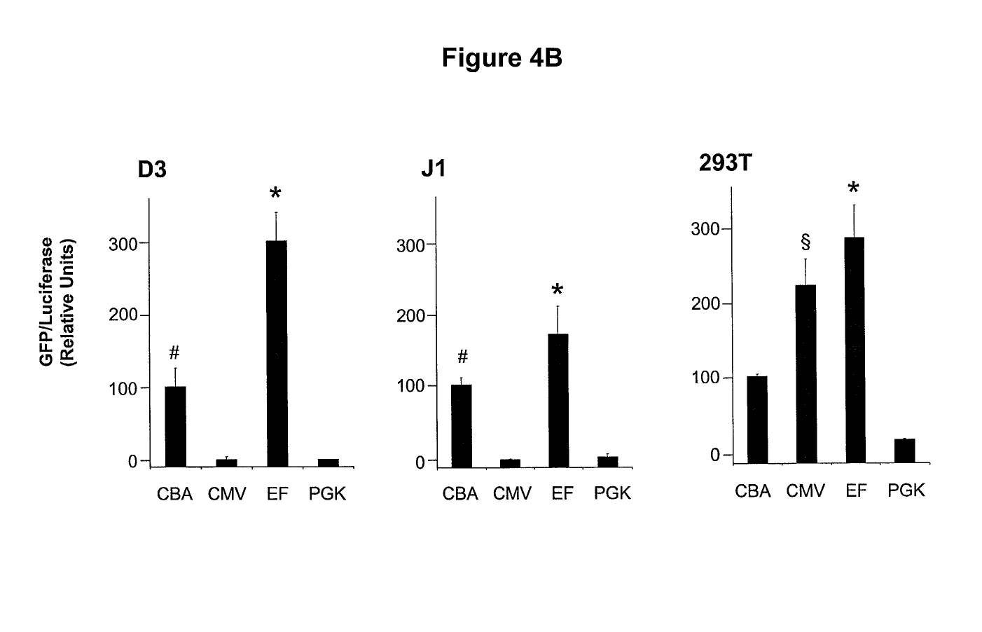

We found that the CMV promoter/enhancer drives only a minimal level (possibly an undetectable level) of expression of the luciferase reporter. PGK promoter was also largely inactive in ES cells. In contrast, EF and CBA promoters were shown to drive reporter expression robustly (FIG. 4). In 293T cells, the CMV promoter was able to drive reporter expression as robustly as any other cellular promoter. Taken together, we conclude that the EF or CBA promoters are good choices for transgene expression in ES cells. One skilled in the art would appreciate that this method may also be routinely used to assay expression from other promoters known in the art, such as to determine the expression of a variety of heterologous genes from different promoters in stem cells. Similarly, direct or indirect detection of expression of a heterologous gene may be used to characterize the relative expression from various known promoters in embryonic stem cells.

### EXAMPLE 9

Isolation of ES cell lines that exogenously express Nurr1 from the EF promoter Nurr1 was selected as an example of a possible regulator of the neural cell fate, specifically the dopaminergic fate because of its specific transactivation of the TH gene. Given that expression of the TH gene is essential for dopaminergic neuron function, identification and genetic modification of such selective transcription factors will be one important means to select candidate cell fate inducing genes for engineering of ES cells. We have studied the function of several candidate transcription factors that may play a key role in TH gene induction. Our site-directed mutational analyses further indicate that Nurr1 can directly activate TH gene transcription via more than one mechanisms with or without direct DNA binding, encouraging characterization of transgenic cells expressing Nurr1from a heterologous promoter.

To generate genetically modified ES cell lines that exogenously express Nurr1 under the control of the EF promoter, we first made a Nurr1-expressing vector using the pEF/IRES/hrGFP plasmid. This construct contains the internal ribosome entry sites (IRES) between the Nurr1 and hrGFP coding region and permits both the Nurr1 and hrGFP gene to be translated from a single bicistronic mRNA. The resultant plasmid, pEF/Nurr1/IRES/hrGFP was confirmed by restriction mapping and sequencing analysis. To generate pEF/Nurr1/IRES/hrGFP plasmid, mouse Nurr1 cDNA was inserted into the SalI and BstEII site of pEF/IRES-hrGFP vector. Additionally, the elongation factor promoter has be used to control expression of mouse Nurr1 in other expression plasmids, and FIG. 3B shows a plasmid map of pIRES2/Nurr1/EGFP, which expresses both enhanced green fluorescent protein (EGFP) and transcription factor Nurr1.

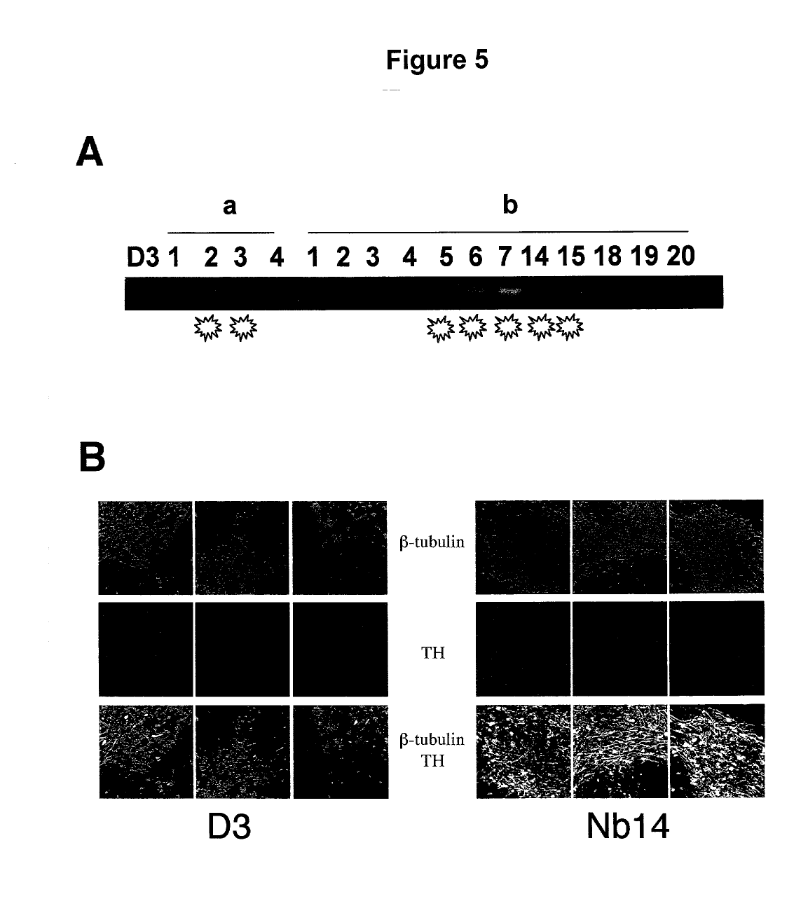

Nurr1-expression plasmid was linearized and used for transfection of D3 cells. Transient cotransfection assays showed that this plasmid transactivates reporter gene expression driven by TH-CAT reporter construct. In an exemplary experiment, the pEF/Nurr1/IRES/hrGFP construct was transfected to D3 cells using Lipofectamin PLUS (GIBCO BRL). Transfected D3 cells were grown on ES media containing 500 μg/ml Neomycin (G418 Sulfate, Clontech). Each Neo^{r} clone was analyzed for Nurr1 expression by RNA preparation and reverse transcriptase PCR analysis. We found that 6 out of 16 clones prominently express Nurr1 niRNA (FIG. 5A).

### EXAMPLE 10

Characterization of cell fate pathway in Nurr1-expressing ES cells

We chose three Nurr1-expressing ES cell lines for further characterization. The naïve D2 cells and Nurr1-expressing cells exhibited similar pattern of formation of nestin⁺ neural progenitor cells. However, we found that all three Nurr1-expressing ES cell lines showed much higher efficiency of TH⁺ positive neurons after in vitro differentiation procedure, compared to the naïve ES cells (FIG. 5B). Furthermore, most of these TH⁺ neurons were shown to be AADC⁺ suggesting that these neurons may have dopaminergic phenotype. Methods for identifying neuron-specific markers used to further characterize the in vitro or in vivo differentiation fate of Nurr1-expressing ES cells are described herein. See, e.g., Example 12.

In FIG. 5B, in vitro differentiated cells are β-tubulin positive (green), and cells positive for the dopaminergic marker, TH, are indicated by red staining.

After in vitro differentiation, many more cells derived from the Nurr1 clone, Nb14, are TH positive, as compared to in vitro differentiated D3 cells. Thus, the Nurr1-expressing ES cells exhibit a higher efficiency of in vitro differentiation to tyrosine hydroxylase-positive cell fate, a well correlated marker for dopaminergic differentiation. This demonstrates an effective method of genetic modification of ES cells to induce the dopaminergic phenotype.

We will further characterize Nurr1-expressing D3 ES cell lines by RT-PCR, Northern and Western blot analyses for dopaminergic marker proteins after in vitro differentiation. We will then use these genetically modified ES cells for transplantation and in vivo differentiation in rodent models of PD, such as those described below.

### EXAMPLE 11

Inducible expression of Nurr1 in ES cells

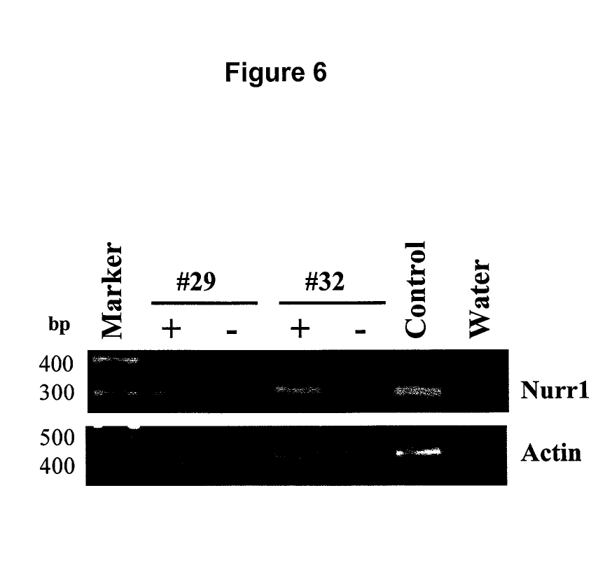

Next, ES cell lines were constructed that express Nurr1 in a tetracycline-inducible manner. To generate transgenic ES cell lines that can express Nurr1 in a regulatable manner, the Nurr1 cDNA was first cloned into the Tet-response vector pTRE2 (Clontech), resulting in pTRE2-Nurr1. The J1-rtTA cell line, which stably expresses the rtTA, is an ideal system for our purposes, because the inducibility of the gene by doxicycline as well as genetic stability of this novel ES cell line have recently been established. (Wutz, A, et al., 2000, Mol. Cell, Vol. 5, 695). Using a Bio-Rad Genepulser set at 25 uF and 400V, we co-transfected J1-rtTA cells with the linearized plasmids pTRE2-Nurr1 (30 μg) and pPGKpuro (3 μg) which expresses the puromycine resistant gene under the PGK promoter. The transfected cells were cultured in stem cell media containing 50 ug/ml LIF and selected in the presence of puromycin (2 μg/ml). From 38 individual colonies picked from the plates, 21 clones were expanded and further analyzed for doxycycline-controlled induction of Nurr1 expression. Doxycycline was treated at 1 μg/ml to the culture media and cells were harvested after 36 hrs. mRNAs were prepared and examined for expression of Nurr1 message by RT-PCR. Oligonucleotides detecting either the Nurr1 (300 bp) or actin (415 bp) transcripts were used for comparison. 7 of the 21 clones initially analyzed (approximately 30%) were found to express Nurr1 upon addition of doxycycline. Two (#29 and #32) of these clones will be used for further analyses. Inducible Nurr1 expression in the stably transfected J1-rtTA-Nurr1 clones #29 and #32 is shown in FIG. 6.

Modulation of timing and degree of Nurr1 induction may effect the DA phenotype determination in vitro and in vivo. Transplantation following various induction protocols will allow optimization of DA differentiation for the various functional responses desired. Characterization of the effects of altering parameters including timing and extent of Nurr1 induction may allow specific generation of more or less homogenous nerve cell populations in the transplant. Other inducible expression systems known in the art may similarly be used to express a heterologous gene in the ES cells of the invention. Numerous inducible systems for modulating gene expression, which increase or reduce expression of target genes, are well known in the art.

### EXAMPLE 12

Effect of transplantation of lower numbers ES cells on cell fate

Donor cell grafts with high cell densities, such as those described in Example 4, create conditions where the majority of cell-cell interactions are between ES cells, not between ES cells and host cells. Alternately, implantation of low cell numbers is featured in the invention. Dilution of ES cells, preferably suspensions of dissociated cells such as single cell suspensions of low ES cell concentrations, facilitates development of neural cells upon transplantation or implantation of the ES cells suspensions in vivo. Grafts of low cell numbers of naïve ES cells develop into normal midbrain-like DA neurons in animal models of Parkinson's Disease.

Low density cell suspensions were prepared essentially as described in Example 1, with the following modifications. Early passage cultures, after culturing for two weeks in the presence of LIF, were trypsinized (0.05% trypsin-EGTA; GIBCO), resuspended, and seeded at 5×10⁶ cells in 15 ml of DMEM plus 10% FCS in a 100 mm Fisher brand bacteriological grade petri dish for 4 days in the absence of LIF. Cells were transferred to a 15 ml sterile culture tube and allowed to settle, spun at 1000 rotations/minute for 5 minutes, then collected and rinsed once in Ca²⁺ and Mg²⁺-free Dulbecco's Phosphate-Buffered Saline (D-PBS, Gibco/BRL). After rinsing, D-PBS was removed and 1.5 ml of trypsin solution was added. The cells were incubated for 5 minutes at 37° C., then triturated with fire polished Pasteur pipettes with decreasing aperture size to fully dissociate the cells. Finally, ES cells were spun at 1000 rotations/minute for 5 minutes, allowing trypsin solution to be replaced with 200 μl culture media, and the viability and concentration of ES cells was determined using a hemocytometer after staining with acridine orange and ethidium bromide.

To examine the in vivo fate of ES cells, mouse ES cell suspensions of low density were grafted into the mouse striatum. The procedures used are essentially as described in Example 4, with modifications as follows. Male C57BL6 nmice (25 g. Charles River, Wilmington, Mass.) were injected intraperitonially (i.p.) With 20 mg/kg MPTP (Research Biochemicals International, Natick, Mass.) twice per day for 2 days (at 12 hour intervals), then once per day for the following 3 days (total MPTP dose=140 mg/kg) as described in Costantini et al Neurobiol. Dis. 5, 97-106 (1998). The mice were transplanted 11 days after the last MPTP injection. The MPTP treatment does not create a complete and permanent DA lesion of the striatum or influence the grafted ES cells, but it facilitates identification of TH-positive neurons in the graft-host interface. Mice were anesthetized with an i.m. injection of a mixture of ketamine (100 mg/kg, Ketaset, Fort Dodge, Iowa) and xylazine (5 mg/kg, Xyla-Ject, Phoenix Pharmaceuticals, St. Joseph, Mo.). Each animal received an injection of 1.0 μl (0.25 μl/min) ES cell suspension into the right striatum using a 22-gauge 10 μl Hamilton syringe. The needle was removed after a two minute wait. The mice were divided into two groups depending on the amount of cells injected (D3 2,000/μl n=5 and 200/μl n=7).

### EXAMPLE 13

Characterization of low cell number transplants

The in vivo fate of ES cell transplants were examined at 4 weeks survival using immunofluorescence and confocal microscopy to identify graft markers in the transplanted cells. In these experiments, 50,000, 2,000 and 200 ES cells were grafted into the striatum of MPTP-treated mice. Cell suspensions ranging from 50,000 to 100 cells per microliter of solution were used. Histological evaluation 4 weeks post-transplantation revealed tumor-like grafts in 6 out of 7 cases when 50,000 ES cells were grafted. When 2,000 or 200 ES cells were grafted, all grafts were non tumor-like and most grafts contained numerous tyrosine hydroxylase (TH) positive neurons with the 200 ES cell grafts producing more TH-positive neurons per cell grafted than the 2,000 cell grafts. The 200 implanted D3 ES cells resulted in an average of 1250 DA neurons and did not produce any tumor-like structure even 8 weeks post transplantation (n=8). These findings indicate that the problem of tumor-like formation may be reduced by decreasing the number of ES cells per graft or by decreasing the concentration of ES cells in suspension (measured in cells/μl pharmaceutically acceptable carrier). Terminal differentiation into a stable non-dividing neuronal pheotype was consistent with the absence of staining against proliferating cell nuclear antigen (PCNA) or the proliferation marker Ki-67 in the differentiated neuronal graft.

Implanted ES cells primarily developed into neural grafts with high numbers of mature ventral midbrain-like DA neurons identified by markers such as TH, AADC, DAT, AHD 2 and calbindin, normally present in adult A9 and A10 DA neurons. In addition to DA neurons, the differentiated ES cell grafts developed numerous 5HT neurons. It is not known how these 5HT neurons will affect the functional properties of the differentiated striatal ES cell grafts. 5HT has been shown to increase synaptic DA release from DA terminals in striatum indicating that the presence of 5HT neurons in our grafts may be beneficial for DA release.

Dopaminergic neuronal phenotypes were demonstrated by co-labeling of DA key proteins such as TH, aromatic amino acid decarboxylase (AADC), and the DA transporter (DAT). ES cell-derived TH-positive neurons were visualized that co-expressed AACD and DAT. Cellular distribution of TH and DAT staining showed very similar patterns, while numerous AADC positive cells were found that did not show immunoreactivity against TH or DAT. We also found ES cell-derived TH-positive neurons co-expressing the A9 midbrain DA neuron marker aldehyde dehydrogenase 2 (AHD 2) or calbindin which is primarily expressed in A10 DA neurons. These findings demonstrate that grafted ES cells differentiate into an adult ventral mesencephalic-like DA neuronal phenotype after transplantation in vivo at low cell densities and dose. The presence of numerous AACD-positive neurons negative for TH or DAT can be explained by the presence of seratonin (5HT) neurons that also coexpress AADC. All TH and 5HT-positive cells expressed the neuronal marker NeuN. To determine if some of the TH-positive neurons in the grafts could be noradrenergic we performed double labeling for TH and DA beta hydroxylase (D H) and we did not find any D H-positive neurons within the grafts. In addition to monoaminergic neurons, grafts also contained a small number of GABA neurons as well as some cholineacetyltransferase (ChAT) neurons.

For histological procedures, animals were terminally anesthetized by an i.p. overdose of pentobarbital (150 mg/kg) four weeks (mice) or 14-16 weeks (rats) after implantation of ES cells, then perfused intracardially with 100 ml heparin saline (0.1% heparin in 0.9% saline followed by 200 ml paraformaldehyde (4% in PBS). The brains were removed and post-fixed for 8 hours in the same solution. Following post-fixation, the brains were equilibrated in sucrose (20% in PBS), sectioned at 40 μm on a freezing microtome and serially collected in PBS.

Multiple labeling fluorescence staining was used for immunohistochemical analysis of the transplants. Sections were rinsed for 3×10 minutes in PBS, preincubated in 4% normal donkey serum (NDS; Jackson Immunoresearch Laboratory, PA) for 60 minutes, and then incubated overnight at room temperature in sheep anti-tyrosine hydroxylase; TH (Pel-Freeze, Rogers, Ark./P60101-0; 1:200), rabbit anti-serotonin (INCSTAR, Stillwater, Minn./#20080; 1:2500), rabbit anti-dopamine beta hydroxylase; DBH (Chemicon, Temecula, Calif./AB145; 1:200), sheep anti-aromatic aminoacid decarboxylase; AADC (Chemicon, Temecula, Calif./AB119; 1:200), rat anti-dopamine transporter; DAT (Chemicon, Temecula, Calif./MAB369; 1:2000), mouse anti-calbindin (SIGMA, St Louis, Mo.; 1:1000), rabbit anti-aldehyde dehydrogenase 2; AHD 2 (a kind gift from Dr. Lindahl; 1:1500), mouse anti-NeuN (Chemicon, Temecula, Calif. /MAB377; 1:200), rabbit anti-GABA, mouse anti-NeuN (1:200) (all from Chemicon, Temecula, Calif., rabbit anti-ChAT (Boehringer Mannheim, Germany, 1:500); mouse anti-PCNA and goat anti-Ki 67 (both from Santa Cruz Biotech. Inc., 1:100), rat anti M6 (Hybridoma Bank, UIOWA, 1:1000) diluted in PBS with 2% NDS and 0.1% Triton X-100. After additional rinsing 3×10 minutes in PBS the sections were incubated in fluorescent labeled secondary antibodies (Cy2/Rhodamine Red-X/Cy5 labeled, raised in donkey; Jackson Immunoresearch Laboratory, PA) in PBS with 2% NDS and 0.1% Triton X-100 for 60 minutes at room temperature. After rinsing, 3×10 minutes in PBS, sections were mounted onto gelatin-coated slides and coverslipped in Gel/Mount (Biomeda Corp. CA). Fluorescence staining was evaluated using a Leica TCS-NT Laser Confocal microscope equipped with argon, krypton/argon and helium lasers. Sections used for TH cell counting was stained using rabbit anti-TH (PelFreeze, Rogers, Ariz., 1:500) and standard ABC technique as described in Deacon, et al., Exp. Neurol. 149, 28-41 (1998). Counting of TH-positive neurons was performed on every 6^{th} section using a Zeiss Axioplan light microscope with a 20× lens. Only stained cells with visible dendrites were counted as TH-positive neurons and the cell counts from serial sections were corrected and extrapolated for whole graft volumes using the Abercrombie method.

### EXAMPLE 14

Transplantation of ES cells in 6-hydroxydopamine (6-OHDA) lesioned rats

Rat experimental models for Parkinson's disease allow functional evaluation of the effects of implantation of ES cells, such as naïve or transgenic cells. Naïve ES cells were implanted in the striatum of 6-OHDA-lesioned rats. First, female Sprague-Dawley rats (200-250 g, Charles River, Wilmington, Mass.) received unilateral stereotaxic injections of 6-OHDA (Sigma, St. Louis, Mo.) into the median forebrain bundle (mfb) as previously described. Costantini, et al., Eur. J. Neurosci. 13, 1085-92 (2001). All coordinates were set according to the atlas of Paxinos.

Next, lesioned animals were selected for transplantation by quantification of rotational behavior in response to amphetamine (4 mg/kg i.p.). Animals were placed (randomized) into automated rotometer bowls and left and right full-body turns were monitored via a computerized activity monitor system. Animals showing >500 turns ipsilateral towards the lesioned side after a single dose of amphetamine were considered having >97% striatal dopaminergic lesion and were selected for grafting. (For example see e.g., Ungerstedt, et al., Brain Research 24, 485-493 (1970))

Rats were given Acepromazine (3.3 mg/kg,PromAce, Fort Dodge, Iowa) and atropine sulfate (0.2 mg/kg, Phoenix Pharmaceuticals, St. Joseph, Mo.) i.m. 20 min before 6-OHDA-lesioned animals were anesthetized with ketamine/xylazine (60 mg/kg and 3 mg/kg respectively, i.m.). Animals were then placed in a Kopf stereotaxic frame (David Kopf Instruments, Tujunga, Calif.). Each animal received an injection of 1.0 μl (0.25 μl/min) ES cell suspension or vehicle into two sites of the right striatum (from Bregma: A+1.0 mm, L−3.0 mm, V−5.0 mm and −4.5 mm, I.B 0) using a 22-gauge, 10 μl Hamilton syringe. All coordinates were set according to the atlas of Franklin and Paxinos. After the injection of cells, 2 min waiting allowed the ES cells to settle before the needle was removed. Animals received 1000-2000 ES cells/μl). After surgery, each animal received an i.p. injection of buprenorphine (0.032 mg/kg) as postoperative anesthesia. Nineteen rats received ES cell injections, and 13 rats received sham surgery by injection of vehicle (media). Five rats died prior to completed behavioral assessment and were found to have teratoma-like tumors at post mortem analysis. A set of 5 rats that did not receive full behavioral testing was analyzed histologically.

To prevent rejection of grafted mouse ES cells, rat hosts received immunosupression by subcutaneous (sc) injections of Cyclosporine A (CsA, 15 mg/kg, Sandimmune, Sandoz, East Hannover, N.J.), diluted in extra virgin oil, given each day starting with a double dose injection one day prior to surgery. Ten weeks post-grafting, dosage was reduced to 10 mg/kg. As a control to examine if immunosupression would affect mouse D3 ES cell graft survival and/or differentiation after transplantation into mice, transplanted mice were divided into two groups with or without immunosupression. CsA was diluted in oil and given each day from the day of surgery as a s.c injection (10 mg/kg). We concluded that CsA treatment does not affect graft survival or differentiation in this experiment.

### EXAMPLE 15

Functional recovery of animal models of Parkinson's Disease

Dopaminergic neurons that develop from transplanted ES cells can restore cerebral function and behavior in animal models of Parkinson's Disease. ES cell derived DA neurons caused gradual and sustained behavioral restoration of DA mediated motor asymmetry.

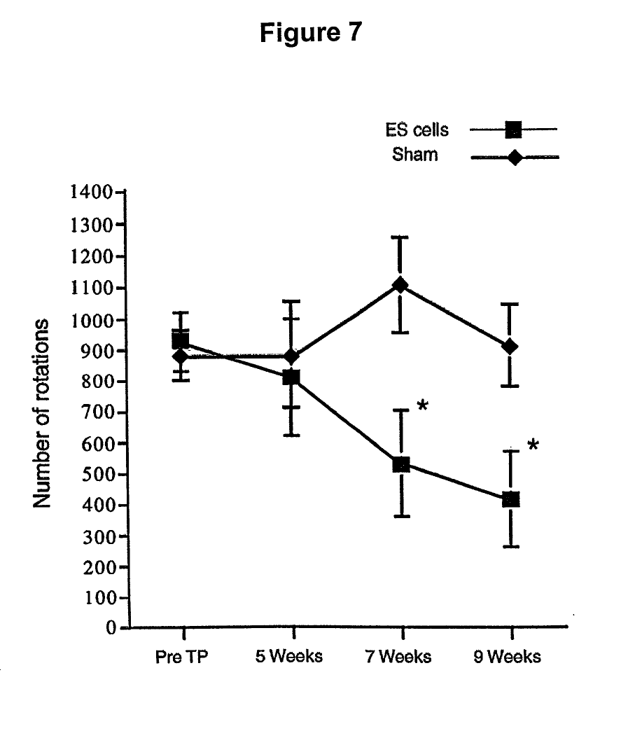

Since the 6-hydroxydopamine (6-OHDA) rat experimental model of dopamine deficiency in Parkinson's disease allows functional evaluation, whereas the mouse does not, we implanted ES cells in the striatum of 6-OHDA-lesioned rats. Lesioned animals were selected for transplantation by quantification of rotational behavior in response to amphetamine. The rotational response to amphetamine was examined at 5, 7 and 9 weeks post-transplantation (FIG. 7). Animals with ES cell derived DA neurons showed recovery over time from amphetamine-induced turning behavior, while control (sham surgery) animals did not (z=3.87, p<0.001). Importantly, decrease in rotational scores was gradual (FIG. 7) and animals with ES cell derived DA neurons showed significant decrease in rotations from pre-transplantation values at 7 weeks and at 9 weeks. Similar significant differences were obtained in measures of percentage change in rotations.

As demonstrated in FIG. 7, mouse ES cells restore DA dependent motor function in 6-OHDA lesioned rat striatum. Rotational behavior in response to amphetamine (4 mg/kg) was tested pre-transplantation (pre TP) and at 5, 7 and 9 weeks post-grafting in this experiment. A significant decrease in absolute numbers of amphetamine-induced turning was seen in animals with ES cell neural DA grafts in the striatum (n=9) compared to control animals that received sham surgery (n=13). Animals with sham surgery showed not change in rotational score over time (t=1.51, p=0.14). In contrast, animals with ES cell derived neural grafts showed a significant reduction in rotations over time (t=−5.16, p<0.001). We then examined at what time point rotational decrease was significantly reduced compared to pre-transplantation scores. Because we performed post-hoc comparisons, Bonferroni correction was applied to the significance criterion (adjusted criterion, p=0.05/3=0.017). At 5 weeks post-grafting, ES cell grafted animals showed no significant difference in rotations compared to pre-transplantation scores (808±188 rotations vs. 924±93 rotations, t=−0.62, p=0.58). However, a clear and significant difference was evident at 7 weeks (530±170 rotation vs. 924±93 rotations, t=−3.66, p=0.0064) and further at 9 weeks (413±154 rotations vs. 924±93 rotations, t=−4.30, p=0.0026). In FIG. 7, * indicates p<0.01.

Additionally, the transplanted cells appear to have functional effects on dykinesias associated with DA deficiency. We demonstrate a progressive and sustained attenuation of dyskinesias in rats with differentiated DA neurons from ES cell transplants. In a preliminary study (n=8) five rats with surviving DA grafts had either a reduction of L-DOPA induced dyskinesias or no change. The development of dyskinesias in parkinsonian patients is thought to result from continuing loss of striatal dopaminergic (DA) terminals. The ES cell-derived transplants alleviate dyskinesias induced in rats with 6-OHDA-induced unilateral nigrostriatal degeneration following administration of 12 mg/kg levodopa/15 mg/kg benserazide (i.p.) twice daily for 3 weeks. Indeed, some grafted animals exhibited no dyskinetic behaviors following challenge with levodopa/benserazide as we observed in rats without 6-OHDA lesions. Thus, DA neurons derived from embryonic stem cells exhibit an ability to reverse neurological disorders (dyskinesis and amphetamine induced rotational behavior) associated with dopaminergic neuron abnormalities.

### EXAMPLE 16

Imaging transplants in Parkinson's disease model

Behavioral recovery paralleled in vivo Positron Emission Tomography (PET) and functional Magnetic Resonance Imaging (fMRI) data, demonstrating DA mediated hemodynamic changes in the striatum and associated brain circuitry. We used PET and carbon-11-labeled 2β-carbomethoxy-3β-(4-fluorophenyl) tropane (¹¹C-CFT) to obtain parallel evidence of DA cell differentiation in vivo. Animals showing behavioral recovery of rotational asymmetry at 9 weeks after implantation of ES cells had an increase in ¹¹C-CFT binding in the grafted striatum of 75-90% (n=3) of the intact side while almost no specific activity (<25% of intact side) was found in controls (n=2).

To study if a gradual functional integration occurs between ES cells derived DA neurons and the host brain in this Parkinson's Disease model, we performed functional magnetic resonance imaging (FMRI) after an amphetamine challenge. Variations in neuronal activity affect the cerebral oxygen consumption rate that can be measured through MRI evaluation of relative cerebral blood flow (rCBV). (For methods, see, for example, Chen, et al., Magn. Reson. Med. 38, 389-98 (1997), and Mandeville, et al., Magn. Reson. Med. 45, 443-7 (2001)). DA release in response to amphetamine induces a specific and significant increase in rCBV in the cortico-striatal circuitry which is coupled to neuronal metabolism. This hemodynamic response is absent following 6-OHDA lesion. ES cell grafted animals (n=4) had a robust activation in response to amphetamine in the grafted striatum and ipsilateral sensorimotor cortex. Significant signal changes in these areas were at similar magnitude to those obtained in the contralateral (non-lesioned) hemisphere. Control animals (sham surgery, n=3) had no response (no signal change) or deactivation (significant decrease) in the same regions. These data support the interpretation of ES cells that become appropriate DA neurons that integrate functionally within the host brain, and provide exemplary methods for functional assessment of transplanted ES cells.

Rats were sacrificed at 14-16 weeks post-transplantation for histological and immunohistochemical analysis. Fourteen animals had grafts located in the striatum. Numerous TH-positive cell bodies (2059+/−626) were identified at the implantation site and TH-positive neurites were found innervating the host striatum. TH fibers close to the graft border had similar density to that seen in the contralateral, non-lesioned host striatum. As expected, all TH-positive cells co-expressed NeuN as well as other DA proteins (DAT, AADC, AHD 2, calbindin). All DA neurons in the rat striatum were labeled by the M6 mouse specific antibody, indicating they were derived from implanted mouse ES cells.

The present invention has been described in terms of particular embodiments found or proposed by the present inventors to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

All publications, patents and patent applications are herein incorporated by reference in their entirety to the same extent as if each individual publication, patent or patent application was specifically and individually indicated to be incorporated by reference in its entirety.

Other embodiments are within the claims.

What is claimed is:

1. A method of treating a human patient suffering from a neurodegenerative disease, said method comprising:
engrafting into said patient a population of recombinant cells comprising one or more cell fate-inducing genes that permit said cells to form neurons in said patient.

2. The method of claim 1, wherein said cell-fate inducing genes are one or more of Nurr-1, PTX3, Phox 2a, AP2, and Shh.

3. The method of claim 1, wherein said cells are made by the steps of:
a) obtaining one or more stem cells,
b) transfecting said one or more stem cells with said one or more cell fate inducing genes,
c) selecting one or more transfectants from step b), and
d) expanding said one or more selected transfectants from step c) to form said population of recombinant cells.

4. The method of claim 3, wherein step d) comprises inducing cell division using a growth factor.

5. The method of claim 4, wherein said growth factor is leukemia inhibitory factor.

6. The method of claim 1, wherein said cells are made by the steps of:
a) obtaining one or more stem cells,
b) expanding said one or more stem cells, and
c) transfecting multiple cells in the expanded cells from step b) with said one or more cell fate inducing genes to form said population of recombinant cells.

7. The method of claim 6, wherein step b) comprises inducing cell division using a growth factor.

8. The method of claim 7, wherein said growth factor is leukemia inhibitory factor.

9. The method of claim 1, wherein said one or more cell fate inducing genes permit said cells to form dopaminergic neurons.

10. The method of claim 1, wherein said recombinant cells are a homogenous cell population of a specific neuronal cell-type.

11. The method of claim 10, wherein said one or more cell fate inducing genes permit said cells to form dopaminergic neurons.

12. A method of treating a human patient suffering from a neurological disease, said method comprising:
engrafting into said patient isolated embryonic stem cells as a suspension of 50 to 5,000 isolated embryonic stem cells per microliter in a pharmaceutically acceptable carrier, such that the concentration of isolated embryonic cells is optimized to promote neuronal cell fate in the patient.

13. The method of claim 12, wherein the suspension comprises 100 to 2,000 isolated embryonic stem cells per microliter in a pharmaceutically acceptable carrier.

14. The method of claim 12, wherein fewer than 10,000 isolated embryonic cells are administered to the patient per administration.

15. The method of claim 14, wherein fewer than 2,000 isolated embryonic cells are administered to the patient per administration.

16. A method of treating a human patient suffering from a neurological disease, said method comprising:
engrafting into the patient a population of isolated embryonic stem cells as a suspension of 50 to 5,000 cells per microliter in a pharmaceutically acceptable carrier, such that the cells form, in the patient, a population of cells in which at least 90% the cells are dopaminergic or seratonergic neurons.

17. The method of claim 16, wherein the population of embryonic stem cells is recombinant, comprising one or more cell fate-inducing genes that permit said cells to form neurons in said patient.

18. The method of claim 17, wherein the cell fate-inducing genes are expressed from a heterologous promoter.
